(19) United States
(12) Patent Application Publication
(10) Pub. No.: US 2007/0203391 A1
(43) Pub. Date: Aug. 30, 2007

(54) System for Treating Mitral Valve Regurgitation

(75) Inventors: Eliot Bloom, Hopkinton, NH (US); Morgan House, Newfields, NH (US); Nasser Rafiee, Andover, MA (US); Michael Finney, Beverly, MA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/678,466

(22) Filed: Feb. 23, 2007

Related U.S. Application Data
(60) Provisional application No. 60/743,349, filed on Feb. 24, 2006.

(51) Int. Cl. A61F 2/02; A61B 17/34

(52) U.S. Cl. 600/37; 606/151; 606/185

(57) ABSTRACT

A system for treating mitral valve regurgitation comprising at least delivery catheters, puncture catheters, and tensioning devices. The devices include tension members linking a proximal anchor and distal anchor that can be constructed from a tubular braded material and have internal reinforcing members. In some embodiments, the anchors and tension members may flex in response to a heart beat. The system can also include temporary anchors so a clinician can review and adjust the vector of the tension member. Delivery catheters can also include temporary anchors to secure the catheter in position. When positioned across the left ventricle of a heart, the device can reduce the lateral distance between the walls of the ventricle and thus allow better coaption of the mitral valve leaflets thereby reducing heart valve regurgitation.

### RELATED APPLICATIONS

This application claims priority under 35 U.S.C. §119(e) from U.S. Provisional Application No. 60/743,349, filed February 24.

### TECHNICAL FIELD

This invention relates generally to medical devices and particularly to a system and method for treating mitral valve regurgitation by reducing the lateral space between the ventricular septum and the free wall of the left ventricle.

### BACKGROUND OF THE INVENTION

The heart is a four-chambered pump that moves blood efficiently through the vascular system. Blood enters the heart through the vena cava and flows into the right atrium. From the right atrium, blood flows through the tricuspid valve and into the right ventricle, which then contracts and forces blood through the pulmonic valve and into the lungs. Oxygenated blood returns from the lungs and enters the heart through the left atrium and passes through the bicuspid mitral valve into the left ventricle. The left ventricle contracts and pumps blood through the aortic valve into the aorta and to the vascular system.

The mitral valve consists of two leaflets (anterior and posterior) attached to a fibrous ring or annulus. In a healthy heart, the mitral valve leaflets overlap during contraction of the left ventricle and prevent blood from flowing back into the left atrium. However, due to various cardiac diseases, the mitral valve annulus may become distended, causing the leaflets to remain partially open during ventricular contraction and thus allowing regurgitation of blood into the left atrium. This results in reduced ejection volume from the left ventricle, causing the left ventricle to compensate with a larger stroke volume. The increased workload eventually results in dilation and hypertrophy of the left ventricle, further enlarging and distorting the shape of the mitral valve. If left untreated, the condition may result in cardiac insufficiency, ventricular failure, and death.

It is common medical practice to treat mitral valve regurgitation by valve replacement or repair. Valve replacement involves an open-heart surgical procedure in which the patient's mitral valve is removed and replaced with an artificial valve. This is a complex, invasive surgical procedure with the potential for many complications and a long recovery period.

Mitral valve repair includes a variety of procedures to repair or reshape the leaflets to improve closure of the valve during ventricular contraction. If the mitral valve annulus has become distended, a common repair procedure involves implanting an annuloplasty ring on the mitral valve annulus. The annuloplasty ring generally has a smaller diameter than the annulus, and when sutured to the annulus, the annuloplasty ring draws the annulus into a smaller configuration, bringing the mitral valve leaflets closer together and providing improved closure during ventricular contraction.

Annuloplasty rings may be rigid, flexible, or have both rigid and flexible segments. Rigid annuloplasty rings have the disadvantage of causing the mitral valve annulus to be rigid and unable to flex in response to the contractions of the ventricle, thus inhibiting the normal movement of the mitral valve that is required for it to function optimally. Flexible annuloplasty rings are frequently made of Dacron® fabric and must be sewn to the annular ring with a line of sutures. This eventually leads to scar tissue formation and loss of flexibility and function of the mitral valve. Similarly, combination rings must generally be sutured in place and also cause scar tissue formation and loss of mitral valve flexibility and function.

Annuloplasty rings have been developed that do not require suturing. U.S. Pat. No. 6,565,603 discloses a combination rigid and flexible annuloplasty ring that is inserted into the fat pad of the atrioventricular groove, which surrounds the mitral valve annulus. Although this device avoids the need for sutures, it must be placed within the atrioventricular groove with great care to prevent tissue damage to the heart.

U.S. Pat. No. 6,569,198 discloses a flexible annuloplasty ring designed to be inserted into the coronary sinus, which is located adjacent to and partially surrounds the mitral annulus. The prosthesis is shortened lengthwise within the coronary sinus to reduce the size of the mitral annulus. However, the coronary sinus in a particular individual may not wrap around the heart far enough to allow effective encircling of the mitral valve, making this treatment ineffective.

U.S. Pat. No. 6,210,432 discloses a flexible elongated device that is inserted into the coronary sinus and adapts to the shape of the coronary sinus. The device then undergoes a change that causes it to assume a reduced radius of curvature and, as a result, causes the radius of curvature of the coronary sinus and the circumference of the mitral annulus to be reduced. While likely to be effective for modest changes in the size or shape of the mitral annulus, this device may cause significant tissue compression in patients requiring a larger change in the configuration of the mitral annulus.

U.S. Patent Application Publication 2003/0105520 discloses a flexible elongated device that is inserted into the coronary sinus and anchored at each end by a self-expanding, toggle bolt-like anchor that expands and engages the inner wall of the coronary sinus. Application WO02/076284 discloses a similar flexible elongated device that is inserted into the coronary sinus. This device is anchored at the distal end by puncturing the wall of the coronary sinus, crossing the intervening cardiac tissue, and deploying the anchor against the exterior of the heart in the pericardial space. The proximal end of the elongated member is anchored against the coronary ostium, which connects the right atrium and the coronary sinus. Once anchored at each end, the length of either of the elongated devices may be adjusted to reduce the curvature of the coronary sinus and thereby change the configuration of the mitral annulus. Due to the nature of the anchors, both of these devices may cause significant damage to the coronary sinus and surrounding cardiac tissue. Also, leaving a device in the coronary sinus may result in formation and breaking off of a thrombus that may pass into the right atrium, right ventricle, and ultimately the lungs, causing a pulmonary embolism. Another disadvantage is that the coronary sinus is typically used for placement of a pacing lead, which may be precluded with the placement of the prosthesis in the coronary sinus.

U.S. Pat. No. 6,616,684 discloses a splint assembly that is positioned transverse the left ventricle to treat mitral valve leakage. In one embodiment, the assembly is delivered through the right ventricle. One end of the assembly is anchored outside the heart, resting against the outside wall of the left ventricle, while the other end is anchored within the right ventricle, against the septal wall. The heart-engaging portions of the assembly, i.e., the anchors, are essentially flat and lie snugly against their respective walls. The length of the splint assembly is either preset or is adjusted to draw the two walls of the chamber toward each other.

The splint assembly may be delivered endovascularly, which offers distinct advantages over open surgery methods. However, the endovascular delivery technique is complicated, involving multiple delivery steps and devices, and requiring that special care be taken to avoid damage to the pericardium and lungs. First, a needle or guidewire is delivered into the right ventricle, advanced through the septal wall, and anchored to the outer or free wall of the left ventricle using barbs or threads that are rotated into the tissue of the free wall.

Visualization is required to ensure the needle does not cause damage beyond the free wall. A delivery catheter is then advanced over the needle, piercing both the septal wall and the free wall of the ventricle. The catheter is anchored to the free wall with balloons inflated on either side of the wall. A tension member is then pushed through the delivery catheter such that a distal anchor is positioned outside the heart. During the catheter anchoring and distal anchor positioning steps, care must be taken to guard against damaging the pericardium or lungs, and insufflation of the space between the myocardium and the pericardial sac may be desirable. A securing band is advanced over the tension member to expand the distal anchor and/or maintain it in an expanded configuration. The catheter is withdrawn, and a second (proximal) anchor is advanced over the tension member using a deployment tool and positioned within the right ventricle against the septal wall. A tightening device then holds the second anchor in a position so as to alter the shape of the left ventricle. Excess length of the tension member is thermally severed prior to removal, again posing some risk to tissue in and around the heart.

Therefore, it would be desirable to provide a system and method for treating mitral valve regurgitation that overcome the aforementioned and other disadvantages.

### SUMMARY OF THE INVENTION

The present invention discloses a system for treating mitral regurgitation. One aspect of the current invention is a system for treating mitral valve regurgitation that includes a delivery catheter. The tensioning devices described herein may be slidably received within a lumen of a delivery catheter. During deployment of the devices, the delivery catheters may be secured and stabilized by a temporary anchor. Additionally, temporary anchors may be used to secure the tensioning device in position so that a clinician can test the tension vector and ensure that the mitral regurgitation is sufficiently reduced.

Another aspect of the present invention is a system that includes a device for treating mitral valve regurgitation, comprising a tension member and proximal and distal anchors. The anchors can be made from tubular braided material, such that they can be configured for catheter delivery to a ventricle and then expanded to a generally planar deployment configuration to rest against the septum or free wall of a heart. The anchors can include struts for reinforcing the generally planar structure after the anchor is deployed. The distal anchor is attached to a distal end of the tension member, and the proximal anchor is attached to a proximal end of the tether.

The device comprises a biocompatible material capable of being preset into a desired shape. Such materials should be sufficiently elastic and flexible that the tension member applies a constant tension force between the anchors, while flexing in response to a heartbeat when the device is positioned across a chamber of a heart. To aid in achieving the correct tension across a heart chamber, devices disclosed herein may include tether locking mechanisms.

Another aspect of the present invention is a system for treating mitral valve regurgitation that includes a catheter having a selectively formable distal section that can be used as a delivery catheter for a septal puncture device and heart valve treatment device. The selectively formable distal section comprises a first curve and a second curve that can be selectively formed by applying tension to a first and second control member. The control members are disposed in a control member lumen and they extend from openings in the distal region of the lumen to a more distal point, where each is affixed to the catheter. Tension is applied to the control members by manipulating adjustment members on the proximal portion of the catheter.

Each curve has an apex and a base, with a control member extending across and defining the base of the curve section. The first curve is formed to have a shape that corresponds to the interior shape of a heart chamber so that the catheter can be braced against the interior wall of the heart chamber. The combination of the curve being braced against the wall and the control member extending across the base of the curve provides a stable support for use when extending the puncture system through the septum.

The two curves operate in generally perpendicular planes, which along with center axis rotation and longitudinal motion provide the capability to direct the distal end of the catheter in a wide range of directions such that the puncture system can extend from the delivery catheter in a desired vector. The curves also allow for a wide range of motion at the distal tip of the catheter for maneuvering the puncture system and treatment systems around obstacles in the heart chamber.

Another aspect of the present invention is a system for treating mitral valve regurgitation that includes the above-described tensioning device and further comprises a catheter for puncturing the septum between the right and left ventricles of a heart. The puncture catheter can also be used to puncture the free walls of a heart for anchor placement.

Another aspect of the present invention is a method of treating mitral valve regurgitation by affecting a mitral valve annulus. A first wall of a chamber of a heart is pierced by a puncture catheter. A distal anchor is engaged with a second wall of the heart chamber. A proximal anchor is engaged with the first wall of the heart chamber. A tension member affixed to and linking the proximal and distal anchors, applies a constant tension force to reduce the lateral distance between the two anchors.

Devices disclosed herein are advantageous over previously disclosed devices in that the braided anchors can help dampen shock to supporting tissues and may have reduced fatigue relative to other devices due to the reinforcing structure contained therein. Additionally, the temporary anchors allow a clinician to review potential vectors for the tension member before permanently emplacing the tension device anchors.

The aforementioned and other features and advantages of the invention will become further apparent from the following detailed description of the presently embodiments, read in conjunction with the accompanying drawings, which are not to scale. The detailed description and drawings are merely illustrative of the invention rather than limiting, the scope of the invention being defined by the appended claims and equivalents thereof.

### BRIEF DESCRIPTION OF THE DRAWINGS

FIG. 1 is a longitudinal cross-section drawing of a heart showing a catheter positioned in one chamber of a heart and a tensioning device deployed in another chamber of the heart according to the current invention

FIG. 2 shows one embodiment of a tension device according to the current invention.

FIG. 3 is a longitudinal cross-section view of the proximal anchor of the device depicted in FIG. 2.

FIG. 4 is a longitudinal cross-section view of the distal anchor of the device depicted in FIG. 2.

FIGS. 5A & 5B depicts an embodiment of an anchor for the tensioning devices disclosed herein

FIG. 6 depicts another embodiment of an anchor for the tensioning devices disclosed herein.

FIG. 7 depicts one of the locking members of the device depicted in FIG. 2.

FIG. 8 shows the operation of one embodiment of locking members used for the devices disclosed herein.

FIG. 9 & FIG. 10 show the operation of a proximal anchor as disclosed herein.

FIGS. 11-13 show one embodiment of a puncture catheter as disclosed herein.

FIGS. 14 & 15 show embodiments of delivery catheters having temporary tissue anchors according to the current invention.

FIGS. 16 & 17 show embodiments temporary tissue anchors according to the current invention.

FIG. 18 depicts an embodiment of a temporary anchor according to the current invention.

FIG. 19 shows an embodiment of a delivery catheter as disclosed herein

### DESCRIPTION OF THE PREFERRED EMBODIMENT

The invention will now be described in detail below by referring to the attached drawings, where like numbers refer to like structures. The present invention discloses a system for treating regurgitation in heart valves. The system is shown and described herein as it would be used to treat regurgitation of the mitral valve. The system includes catheters for navigating through the vasculature to chambers of a heart. The catheters can be used for delivering devices for treating heart valve regurgitation. The system also includes catheters for puncturing the wall of a heart chamber.

The catheter is delivered to the heart by passing it through the venous system. This may be accomplished by inserting the catheters into either the jugular vein or the subclavian vein and passing it through the superior vena cava and into the right atrium. Alternatively, the catheter may be inserted into the femoral vein and passed through the common iliac vein and the inferior vena cava into the right atrium. Catheters of the current invention can be delivered through the vasculature to the heart using over-the-guidewire techniques, or they can be delivered without the use of a guidewire. The procedure may be visualized using fluoroscopy, echocardiography, intravascular ultrasound, angioscopy, or other means of visualization.

Also included in the system are devices for treating heart valve regurgitation by applying lateral tension across a chamber of a heart. Various embodiments of devices of the current invention may be referred to herein simply as “the device” or the “tensioning device” and both terms are to be understood to mean the same thing herein.

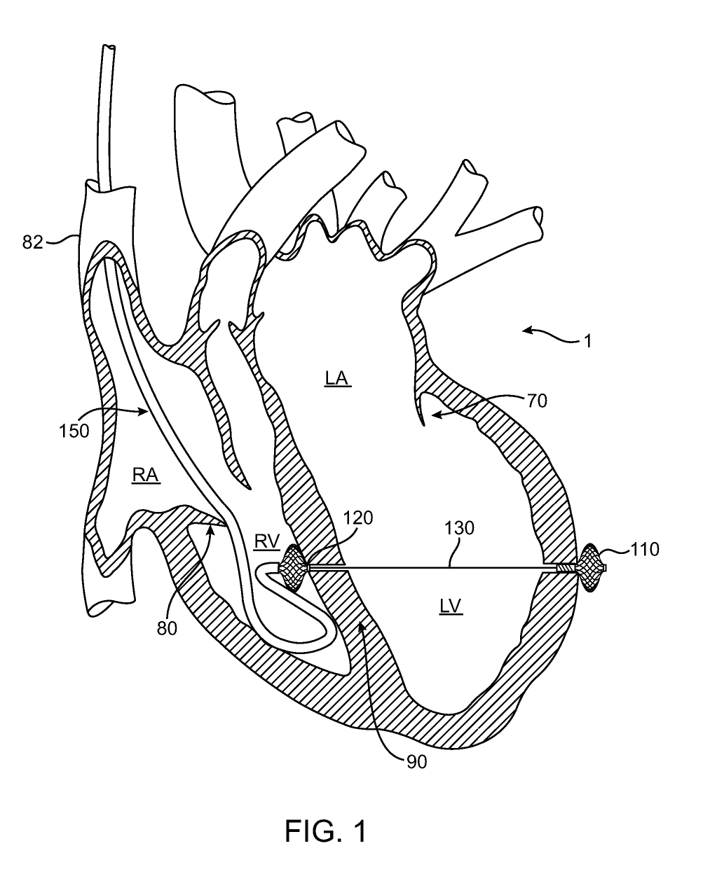

FIG. 1 shows a device for treating mitral valve regurgitation deployed in the left ventricle of a heart according to the current invention. Delivery catheter 150 carrying a tensioning device is passed through the venous system and into a patient's right ventricle. In this case, the delivery catheter has been inserted into either the jugular vein or the subclavian vein and passed through superior vena cava 82 into right atrium RA, and then passed through tricuspid valve 80 into right ventricle RV.

The delivery catheter 150 depicted in FIG. 1 has a selectively formable distal section that can be manipulated to form a first curve and a second curve. The first curve with a shape that corresponds to the inside shape of the right ventricle of a heart so that the catheter can be braced against opposing walls inside the chamber while the septum is being punctured and devices for treating diseased heart valves are being delivered to the heart tissue. The second curve is formed so that the distal tip is oriented such that devices for treating diseased heart valves can be deployed along the proper vector relative to the valve being treated. Catheters having selectively formable distal sections are depicted in U.S. patent application Ser. No. 11/277,062, titled “Catheter Having a Remotely Formable Distal Section,” filed on Mar. 21, 2006, the contents of which are incorporated herein by reference thereto.

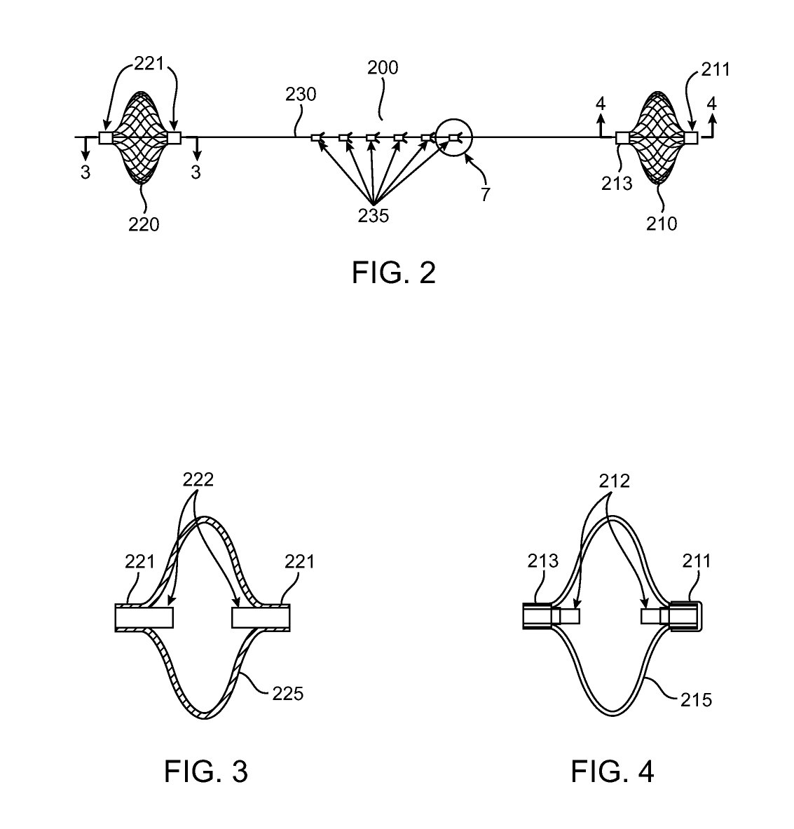

The tension member 130 is extended across the left ventricle, the proximal anchor 120 is deployed in the right ventricle such that it rests against the septum on the right ventricular side of the septum, and the distal anchor 110 is deployed on the outside of the free wall of the heart chamber. The device depicted in the embodiment is the device shown in FIG. 2 and described below.

One embodiment of a device for treating heart valve regurgitation, in accordance with the present invention, is illustrated in FIG. 2, which shows the device in a deployed/deployment configuration as opposed to a delivery configuration, in which the device is in a collapsed state in a catheter.

The representative tensioning device 200 depicted in FIG. 2 is designed to be positioned across a chamber of a heart using catheterization techniques while the heart is beating. The devices disclosed herein can also be delivered and positioned using minimally invasive surgical techniques on a beating heart, or surgical techniques on a heart in which the beating has been temporarily halted. Although described below in the context of treating mitral valve regurgitation by reducing or limiting lateral distension of the left ventricle as the heart beats, the devices of the current invention may be deployed at other locations in the heart and they are readily adapted to a wide variety of uses, including treating ischemic or dilated cardiomyopathy. An example of another location for deployment of a device according to the current invention includes deploying a tension member across the right ventricle of a heart to address regurgitation in a tricuspid valve.

As can be seen in FIG. 2, the device 200 includes a proximal anchor 220 that can be positioned on a proximal end of a tension member 230, and a distal anchor 210 positioned on the distal end of the tension member 230. In one embodiment, the tension member 230 is affixed to an end hub 211 of the distal anchor and it can move freely through an inside hub 213 on the distal anchor and two hubs 221 on the proximal anchor. A plurality of locking members 235 are attached to the tension member at intervals along a portion of the member. When the tension device 200 is deployed, the locking members 235 can be withdrawn proximally, through the proximal anchor 220 and the design of the locking members 235 prevents them from being able to pass back through the proximal anchor 220. As used herein, the terms “distal” and “proximal” refer to the location of the referenced element with respect to the treating clinician during deployment of the device with proximal being closer to the treating clinician than distal.

The tension members of the devices disclosed herein can be constructed from a material having sufficient elastic properties, or constructed in a shape such that the tension member can become elongated (flex) in response to a heartbeat when the device is positioned across a heart chamber and then contract. The distal and/or proximal anchors of the devices disclosed herein can be constructed of a material that will allow the anchors to flex in response to a heart beat. Flexing of the tension member with or without an additional flexing of anchors, reduces the risk of the device failing due to structural fatigue, and also reduces localized compressive pressure on tissue against which the anchors rest.

In the various embodiments described herein, the device comprises a biocompatible material capable of being pre-set into a desired shape. Such materials include, but are not limited to, a nickel-titanium alloy, a nickel-cobalt alloy, another cobalt alloy, a thermoset plastic, stainless steel, a suitable biocompatible shape-memory material, a suitable biocompatible super elastic material, combinations thereof, and the like. In some embodiments, the devices can be constructed from wires of such materials and in others; the devices can be braided from such materials.

In one embodiment of the current invention, the anchor members of the current invention can be formed of a tubular braid of any biocompatible material that will provide suitable strength and flexibility. As can be seen in FIG. 2, end hubs 211, 213, & 221 can be attached to the braided material to prevent the anchor members from unraveling and to allow the tension member to slide freely through the anchors, where it is not attached to the anchors. In a delivery configuration, the tubular braided anchors have a relatively small outer diameter to allow them to pass through a delivery catheter or other delivery member. Once the anchors are deployed, they can assume a deployment configuration where a portion of the tubular braid expands radially outward such that the deployed anchor has a larger outside diameter than it had in a delivery configuration.

According to the current invention, there are a plurality of ways to make the anchors assume a deployed configuration after delivery. In one embodiment, the anchors can be made from a shape memory material and then pre-set in a deployment configuration before being forced into, and restrained in, a delivery configuration. In other embodiments of tension devices, the anchors can be mechanically forced into the deployment configuration after delivery to a heart chamber.

Referring now to FIGS. 3 and 4, there can be seen longitudinal cross-section views of the proximal and distal anchors respectively, taken along line 3-3 and 4-4 of FIG. 2. The proximal anchor includes tubular members 222 disposed inside the anchor hubs 221. The distal anchor includes tubular members 212 disposed inside of the hubs 211 & 213 of that anchor. The tubular members in the proximal anchor of the depicted embodiment have larger inside diameters than the tubular members in the distal hub, because the locking members must be able to pass through the tubular members in the proximal anchor.

The tension member 230 of the device depicted in FIG. 2 is secured inside of the tubular member 212 in the outside hub 211 of the distal anchor. The tension member 230 can slide freely through the tubular member 212 in the inside hub 213 of the distal anchor so that the anchor can be collapsed into the elongated delivery configuration. The tension member can also slide freely through the tubular members 222 in both hubs 221 of the proximal anchor so that anchor can be collapsed and so that the locking members can be drawn through the anchor as well.

Other embodiments of the anchors can include support members made from shape memory materials. The support members can be preset in a deployment configuration and then placed in a delivery configuration. The struts are then restrained in the delivery configuration, and when the device is deployed the restraints are removed so that the struts assume a deployment configuration. Those skilled in the art of making devices from shape memory materials will understand that other techniques exist that would be equally suitable for constructing the struts or anchors such that they will transform from a delivery configuration to a deployment configuration upon delivery.

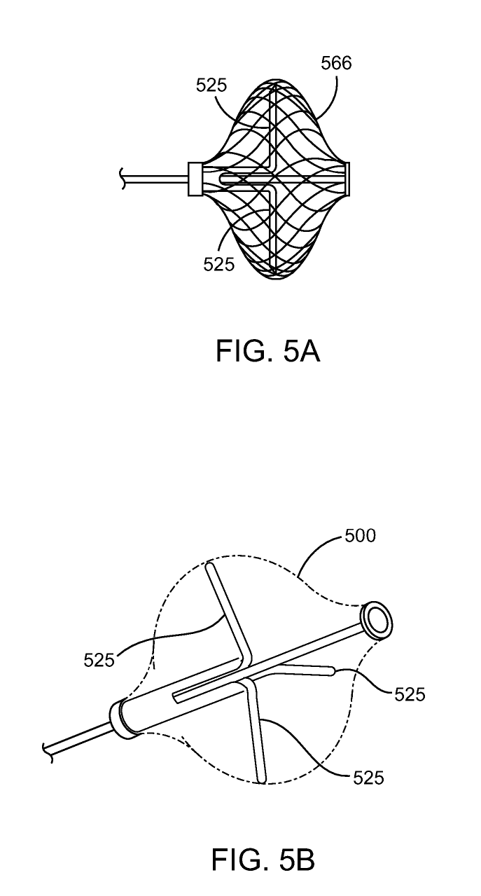

Referring again to FIGS. 3 & 4, the proximal anchor includes struts 225 and the distal anchor has struts 215. Referring now to FIGS. 5A and 5B, there can be seen an embodiment of braided anchor having struts according to the current invention. FIG. 5A shows the braided anchor 500 having struts 525 enclosed therein. FIG. 5B is a schematic showing the struts of the anchor in FIG. 5A with the braided material removed for illustrative purposes. The depicted embodiment has three struts that are arranged to extend radially from the center of the anchor. When the anchor is deployed and rests against the surface of the heart tissue, the struts provide added support to prevent anchor migration through the heart tissue.

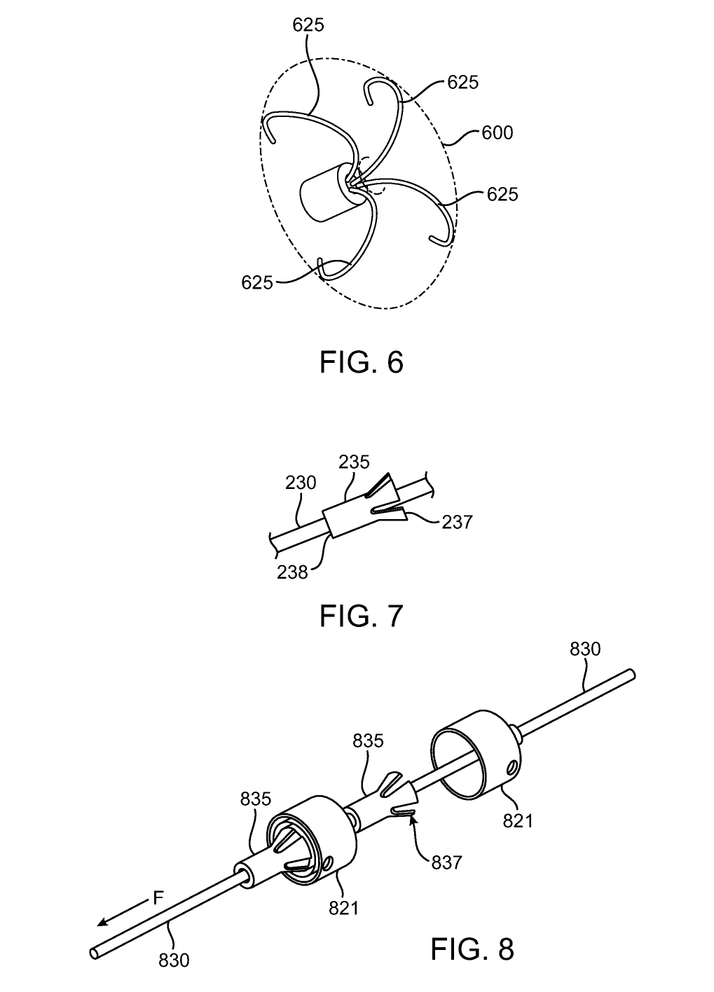

FIG. 6 shows another embodiment of an anchor device having a slightly different strut configuration. The figure shows an anchor 600 with the braided material removed for illustrative purposes. The struts 625 are bowed and a small foot portion of the strut rests against the braided material that engages the heart tissue. The bowed configuration of the struts allow them to deform and recoil during the normal heart cycle.

The intent of the struts is to provide additional structural support and strength to the braided anchors. The anchors can be constructed in two parts and the struts can be placed on the inside of the braided anchor tubing or attached to the outside of the anchor. The struts can be constructed to be rigid, semi-rigid, and/or flexible. The struts provide additional support to the braided anchors without increasing the diameter of the deployed anchor. The struts may assist the anchor in achieving the deployed configuration, and they may work to reduce fatigue on the anchor and dissipate force on the tissue.

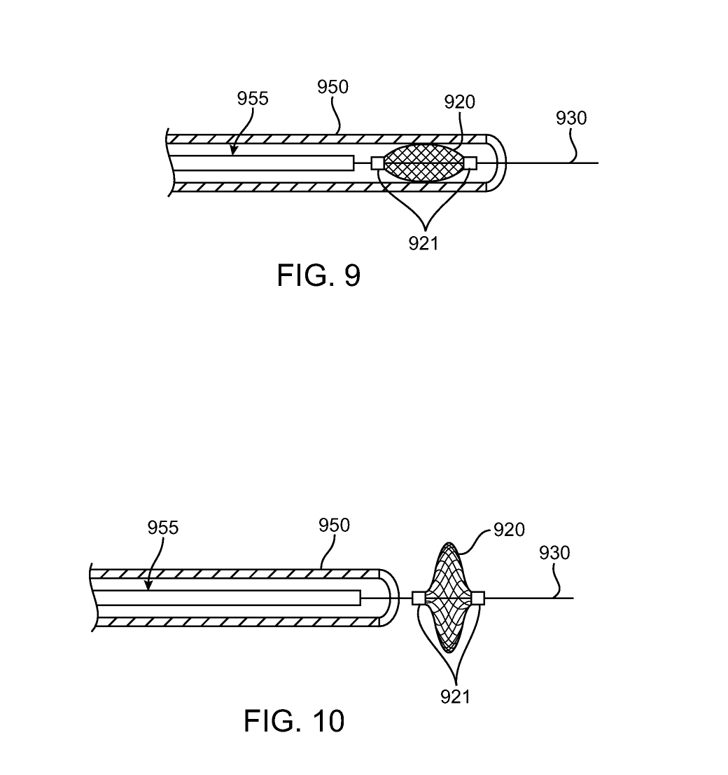

Together the strut and braided anchor combination has the ability to collapse for delivery, and expand after being expelled from a delivery device. Thus the anchors can meet diameter requirements needed for delivery via catheter while still being expandable to provide sufficient resistive support to the tension member so that the devices disclosed herein can properly address mitral regurgitation. This is illustrated in FIGS. 9 and 10. FIG. 9 shows a braided proximal anchor 920 having a pair of hubs 921 in a collapsed delivery configuration inside of the lumen of a catheter 950. Referring to FIG. 10, after the anchor has been expelled from the catheter by a pushrod 955 it expands. After the anchor has expanded, the tension member 930 is drawn through the anchor until the desired length of the member is achieved and the locking mechanisms (not shown) have been engaged.

FIG. 7 shows an enlarged view of one of the locking members 235 that are spaced along the tension member 230 of the device shown in FIG. 2. The locking member is an essentially tubular structure that is attached to the tension member, and it has a plurality of integral legs 237 that extend at an angle from the distal end of the member. The locking member can be tapered such that the outer diameter of the member at its proximal end 238 is smaller than the outer diameter of the member at more distal locations.

Referring now to FIG. 8, there can be seen a partial view of a proximal anchor of the devices shown herein with the braided structure removed so that one can see the interior of the anchor. The hubs 821 each have a lumen communicating therethrough and the hubs could also contain tubular members as described above. After the device has been deployed and the proximal anchor has assumed a deployment configuration, the tension member can be withdrawn through the hubs. A force F is then applied to pull the tension member in a proximal direction, thereby causing at least one of the locking members 835 to be pulled through the hubs. The locking members are made from material having suitable flexibility to allow the legs 837 to compress radially inward when passing through the hubs in a proximal direction and then recoil radially outward so that they will not pass distally through the hubs.

In one embodiment of the invention, if a clinician determines that too much of the tension member has been withdrawn through the proximal anchor, a delivery sheath or similar device can be passed over the locking members to compress the legs inward. The sheath is then moved distally through the proximal anchor until the locking members are distal of the proximal anchor, at which time the sheath is withdrawn.

When positioned across a heart chamber, the anchors and tether are under continuously varying tension due to the motion of the beating heart. To withstand this environment, the tension member may comprise an elastic, biocompatible, metallic or polymeric material that combines elasticity, flexibility, high strength, and high fatigue resistance. For example, the device may be formed using metallic wire, metallic tubes, polymer braid, polymer thread, elastomeric monofilament, elastomeric yarn, etc, so long as the material has suitable elastic properties to allow the tension member to apply a continuous tension force between the two anchor members.

In order to resist excessive elongation during diastole, the material used should stiffen dramatically when elongated. During systole, the tension member should again be elastic to as to recover or recoil. In some embodiments of the invention, it may be desirable to have some pre-load on the tension member to insure that the anchors remain seated and to insure that no slack develops in the tether.

In at least one embodiment, the distal anchor is integral to the tension member. The proximal anchor can be fixedly attached after the correct vector is determined and tension is placed on the tension member to adjust the device to the correct length.

In some embodiments, an antithrombotic component may be included in the chemical composition of a polymeric filament. Alternatively, a polymeric or metallic tether may be coated with a polymer that releases an anticoagulant and thereby reduces the risk of thrombus formation. If desired, additional therapeutic agents or combinations of agents may be used, including antibiotics and anti-inflammatories.

To ensure proper positioning, it is desirable that tensioning device be visible using fluoroscopy, echocardiography, intravascular ultrasound, angioscopy, or another means of visualization. Where fluoroscopy is utilized, any or all of tensioning device may be coated with a radiopaque material, or a radiopaque marker may be included on any portion of the device that would be useful to visualize.

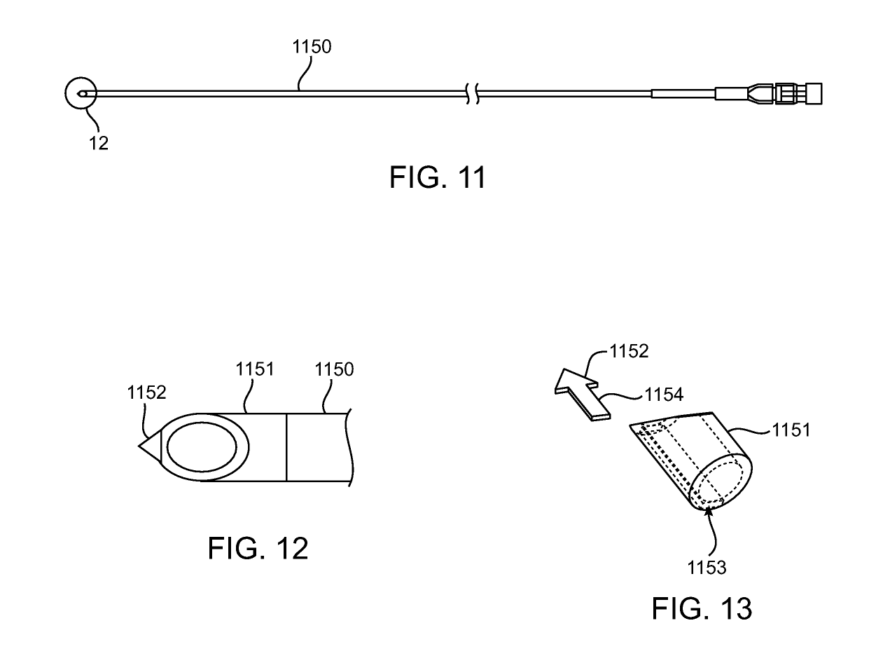

The devices of the current invention may be delivered to the chambers of the heart via catheters having tips for puncturing the heart walls. One example of such catheters can be seen in FIGS. 11-13. The catheter has at least one lumen communicating longitudinally therethrough and it is constructed from material and designed such that it will have sufficient rigidity to allow it to be pushed or rotated through the walls of the heart. The depicted catheter includes a tip 1151 that is attached to the end thereof. The tip includes a slot or channel 1153 communicating therethrough. The channel 1153 is dimensioned such that its shape is complementary to a tang 1154 on a pointed cutting element 1152. The tang is secured in the channel such that the cutting element is secured to the tip, which is secured to the distal end of the catheter.

At least on embodiment of the current invention includes a puncture catheter having a more concentric tip and other embodiments of the invention can include a puncture catheter formed from a sharpened hypo tube. The puncture catheters can access the chambers of a heart via the delivery catheter. Embodiments of puncture catheters of the current invention can also include lumens for injecting contrast medium or therapeutic substances into the heart. Catheters may include aspiration lumens for aspirating blood from the pericardial sac. At least one embodiment of the current invention includes a catheter with a pressure monitoring lumen. One embodiment of the invention includes catheters that are capable of delivering electrical energy to a heart chamber.

Referring again to FIG. 11, in one embodiment of the current invention, the pointed distal tip of the catheter can be placed against the septum and a force can be applied to push the tip through the septum while the catheter is being rotated. When properly executed, this action creates a channel through the septum without removing tissue. The device can then be delivered through the puncture catheter, or the puncture catheter can be temporarily anchored in place. A separate delivery device can then be passed through the lumen in the puncture catheter and used to emplace the tensioning device.

Selecting the proper vector for emplacing the tension member of the current devices is one of the keys for successfully treating mitral regurgitation. It is imperative that the devices disclosed herein and the members used to deliver these devices, do not puncture significant vessels or other significant structure during deployment. Additionally, the tension members of the current invention should be oriented correctly relative to the leaflets of the mitral valve so that the device can achieve best results possible.

One way to determine whether the correct vector has been selected is to extend the device across the heart chamber and secure the proximal and distal ends of the device with temporary anchors. The length of the tension member is then adjusted so that a clinician can check for suitable reduction of mitral regurgitation. If the device is properly positioned, the temporary anchors are withdrawn and permanent anchors are emplaced. If the device needs to be moved, the temporary anchors are collapsed and the device is repositioned until the correct vector is achieved. In at least one embodiment, the temporary anchors can be used to close openings in a myocardium when the device is moved due to incorrect placement.

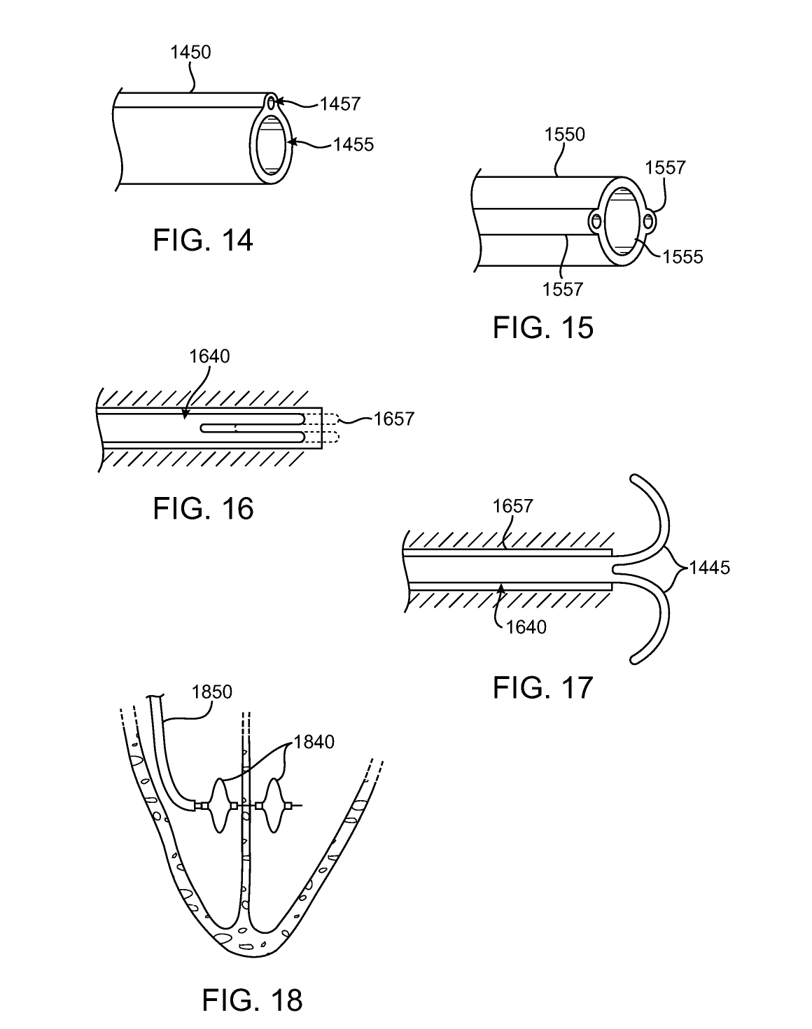

FIGS. 14-18 show several embodiments of devices that can be used for temporarily anchoring the proximal end in position while checking to make sure the proper vector was selected. FIG. 14 shows a catheter 1450 having a delivery lumen 1445 through which the tensioning device can be delivered. The catheter also has an anchor lumen 1457 that can contain a device for temporarily securing the catheter in position while the tensioning device is being deployed. FIG. 15 shows a delivery catheter having two anchor lumens, and other embodiments of the current invention can include delivery catheters having more than two anchor lumens.

FIGS. 16 and 17 show a temporary anchor according to the current invention. The temporary anchors 1640 are made from a biocompatible material having shape memory properties. The depicted embodiment of anchor 1640 is and essentially elongated member having a pair of tissue engaging legs 1455. The anchor is made of a material that will allow the legs to assume a deployment configuration after the anchor is extended from the anchor lumen, such that the legs engage the heart tissue in a manner that secures the catheter in position. In the depicted embodiment, the anchor is forced from the anchor lumen such that the legs are driven into the heart tissue and the legs turn outwardly from the catheter in a bowed configuration and engage the heart tissue. The anchor must be made from a material that will allow the legs to easily collapse back into the delivery configuration when the anchor is withdrawn into the anchor lumen of the catheter.

FIG. 18 shows another embodiment of a temporary anchoring system according to the current invention. The temporary system comprises two braided anchors 1450 (balloons can also be used) that are deployed on opposite sides of the septum such that they temporarily secure the distal end of the delivery catheter in position while the clinician checks the vector of the tension member. In this embodiment, the temporary anchors have lumens large enough to allow delivery devices to pass through the anchors.

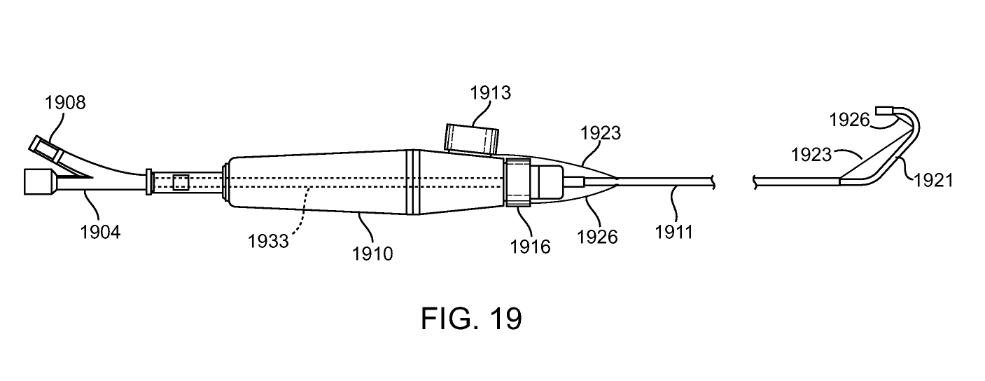

FIG. 19 shows an embodiment of a delivery catheter that can be used in the system of the current invention. The catheter 1901 comprises a handle 1910, a proximal section 1911, and a distal section 1921. As used herein, the term proximal means the portion or end of the catheter that is closest to the clinician manipulating the catheter when it is in use and distal means that portion or end of the catheter that is further away from the clinician when the catheter is in use. The proximal section of the catheter is the portion that is forward or distal of the handle but proximal of the midpoint of the catheter and the distal section of the catheter is that portion that is distal of the proximal section.

The handle 1910 has an input port 1904 and an injection port 1908. A lumen 1933 runs through the handle and along the length of the catheter through the proximal section 1911 and the distal section 1921 before terminating in an opening at the distal tip 28. The lumen 1933 can be used for delivering septal puncture systems or systems for treating heart valve disease to the chambers of a heart.

A first control member 1923, and a second control member 1926 are disposed in the control member lumen. The proximal end of the first control member 1923 extends from an opening in control member lumen that is located in the proximal section 1911 of the catheter and it is connected to a first adjustment member 1913. The proximal end of the second control member 1926 extends from an opening in control member lumen that is located in the proximal section 1911 of the catheter and it is connected to a second adjustment member 1916. the control members of the depicted embodiment can be made from any appropriate biocompatible material including line made from braiding polymeric fibers, and in one preferred embodiment the line is made from polyethylene fibers. Other materials are also suitable for making the control members including braided and single strand metal wires.

The first control member 1923 extends distally from a first opening near the distal end and it is affixed to the catheter at an anchor point that is distal of the opening and proximal of the distal tip. The second control member 1926 extends from a second opening in the control member lumen that is distal of the first opening. The second control member is affixed to the catheter at a second anchor point that is distal of the second opening.

The distal section 1921 of the depicted catheter is selectively formable into a first curve by selectively manipulating the first adjustment member 1913 to apply tension to the first control member 1923 such that the first anchor point is drawn toward the first opening and a first curve is formed. The first control member defines the base of the curve by spanning the space between the first anchor point and the first opening. A second curve can be formed by selectively manipulating the second adjustment member 1916 to apply tension to the second control member 1926 such that the second anchor point is drawn toward the second opening and a second curve is formed. The second control member defines the base of the curve by spanning the space between the second anchor point and the second opening.

In the depicted embodiment, tension is applied by rotating the adjustment members, which causes the control member to wind around a base of the adjustment member. Other embodiments of the invention can use other methods of operation for the adjustment members while applying tension to the control members.

In the depicted embodiment, the first curve can be formed in a shape that is complementary to the inside shape of the right ventricle of a heart so that the catheter can be braced against opposing walls inside the chamber while the septum is being punctured and devices for treating diseased heart valves are being delivered to the heart tissue. In another embodiment, the first curve has a shape that is complementary to the interior of the right atrium such that the curve can be braced against opposing heart walls above the tricuspid valve annulus while the septum is being punctured and devices for treating diseased heart valves are being delivered to the heart tissue. The second control member is manipulated to selectively form the second curve so that the distal tip is oriented such that devices for treating diseased heart valves can be deployed in the proper direction relative to the catheter. The control members extend across the base of the curves to provide additional stability and support when treatment devices are being deployed from the catheter.

To deliver the devices disclosed herein, a catheter can be passed through the vasculature so that it is in the right ventricle. The delivery catheter or a separate puncture device can then be used to puncture the septum and temporary anchors can be used to secure the catheter in position. The device can then be extended across the left ventricle while the clinician images the heart to make sure that the device will be installed having the correct orientation. Once the proper vector is selected, a needle, puncture catheter or other suitable device is used to puncture the free wall of the ventricle and the distal anchor is emplaced using a suitable delivery member. The temporary anchors can then be withdrawn, and the tension member extended across the left ventricle and through the septum. The proximal anchor is deployed and held in place while tension is applied to the tension member. The locking devices pass through the hubs on the proximal anchor member and recoil to prevent distal movement of the devices. The clinician can recheck the vector and check for proper tensioning of the device. The excess can be trimmed from the proximal end of the tension member and the delivery devices can be withdrawn.

The device has been described above in respect to catheter delivery via the right ventricle. It will be apparent to those skilled in the art that the devices may be delivered via the aorta or by other methods. Those skilled in the art will also understand that the tension devices and methods disclosed herein are equally suited for delivery to a beating heart via minimally invasive surgery and delivery to a temporarily halted heart via surgical, minimally invasive surgical, and catheter based delivery. The devices can be delivered by puncturing the free wall of both ventricles and the septum, or by puncturing the free wall of one ventricle and the septum.

While embodiments of the invention have been disclosed herein, various changes and modifications can be made without departing from the spirit and scope of the invention. The scope of the invention is indicated in the appended claims, and all changes and modifications that come within the meaning and range of equivalents are intended to be embraced therein.

What is claimed is:

1. A system for treating heart valve regurgitation, comprising:
a delivery catheter;
means for penetrating the walls of a heart chamber;
a device for treating heart valve regurgitation by applying tension across a hear chamber to reduce the lateral distance between two walls of the chamber; and
means for temporarily securing the delivery catheter in place within a heart chamber while the device for treating heart valve regurgitation is placed in a heart chamber.

2. The system of claim 1 wherein the delivery catheter has a selectively formable distal section that can be formed into at least two curved portions.

3. The system of claim 2 wherein the wherein the shape of one of the at least two curved portions is complementary to the interior shape of a chamber of a heart such that the delivery catheter can be braced against the walls of a heart chamber on opposite sides of the chamber.

4. The system of claim 2 wherein the directional orientation of the distal end of the delivery catheter can be changed by manipulating at least one catheter control member.

5. The system of claim 1 wherein the means for penetrating the walls of a heart chamber is a puncture catheter.

6. The system of claim 1 wherein the device for treating heart valve regurgitation comprises:an elongated tension member;
a braided distal anchor member attached to a distal end of the tension member;
a braided proximal anchor member; and
means for securing the tension member such that it is fixedly attached to the proximal anchor member. The system of claim 6 wherein the anchors are constructed from a tubular braided section of material.

7. The system of claim 6 wherein the anchors are made from a material selected from a group consisting of a nickel-titanium alloy, a nickel-cobalt alloy, a cobalt alloy, a thermoset plastic, stainless steel, a biocompatible shape-memory material, a biocompatible superelastic material, and a combination thereof.

8. The system of claim 6 wherein at least one of the braided anchor members further comprises a plurality of flexible struts.

9. The system of claim 1 wherein the means for temporarily securing the delivery catheter in place within a heart chamber is a temporary anchor.

10. The system of claim 10 wherein the delivery catheter has an anchor lumen and the temporary anchor is an elongated section of shape memory material.

11. The system of claim 11 wherein a distal end of the anchor can be extended from the delivery catheter into the wall of a heart chamber and the anchor can assume a shape that temporarily prevents it from being extracted from the wall of the heart chamber until the anchor is withdrawn into the delivery catheter.

12. The system of claim 10 wherein the temporary anchor comprises at least two braided anchor members.

13. The system of claim 1 wherein at least a portion of the device for treating heart valve regurgitation includes a therapeutic agent selected from a group consisting of an antithrombotic, an anticoagulant, an antibiotic, an anti-inflammatory, and a combination thereof.

14. A system for treating mitral valve regurgitation, comprising:
a delivery catheter having a selectively formable distal section that can be formed into at least two curved portions;
a puncture catheter;
a device for treating mitral valve having an elongated tension member,
a braided distal anchor member attached to a distal end of the tension member;
a braided proximal anchor member, and means for securing the tension member such that it is fixedly attached to the proximal anchor member; and
at least one temporary for temporarily securing the delivery catheter in place within a heart chamber.

15. The system of claim 15 wherein the shape of one of the at least two curved portions is complementary to the interior shape of a chamber of a heart such that the delivery catheter can be braced against the walls of a heart chamber on opposite sides of the chamber and directional orientation of the distal end of the delivery catheter can be changed by manipulating at least one catheter control member.

16. The system of claim 15 wherein at least one of the braided anchor members further comprises a plurality of flexible struts.

17. The system of claim 1 wherein at least a portion of the device for treating mitral valve regurgitation includes a therapeutic agent selected from a group consisting of an antithrombotic, an anticoagulant, an antibiotic, an anti-inflammatory, and a combination thereof.
